SEQUENCE LISTING (13 sequences)

SEQ ID NO: 1 - protein, 367 aa, Homo sapiens
MIRNWLTIFILFPLKLVEKCESSVSLTVPPVVKLENGSSTNVSLTLRPPLNATLVITFEITFRSKNITILELPDEVVVPPGVTNSSFQVTSQNVGQLTVYLHGNHSNQTGPRIRFLVIRSSAISIINQVIGWIYFVAWSISFYPQVIMNWRRKSVIGLSFDFVALNLTGFVAYSVFNIGLLWVPYIKEQFLLKYPNGVNPVNSNDVFFSLHAVVLTLIIIVQCCLYERGGQRVSWPAIGFLVLAWLFAFVTMIVAAVGVITWLQFLFCFSYIKLAVTLVKYFPQAYMNFYYKSTEGWSIGNVLLDFTGGSFSLLQMFLQSYNNDQWTLIFGDPTKFGLGVFSIVFDVVFFIQHFCLYRKRPGYDQLN

SEQ ID NO: 2 - protein, 400 aa, Homo sapiens
MIRNWLTIFILFPLKLVEKCESSVSLTVPPVVKLENGSSTNVSLTLRPPLNATLVITFEITFRSKNITILELPDEVVVPPGVTNSSFQVTSQNVGQLTVYLHGNHSNQTGPRIRFLVIRSSAISIINQVIGWIYFVAWSISFYPQVIMNWRRKSVIGLSFDFVALNLTGFVAYSVFNIGLLWVPYIKEQFLLKYPNGVNPVNSNDVFFSLHAVVLTLIIIVQCCLYERGGQRVSWPAIGFLVLAWLFAFVTMIVAAVGVTTWLQFLFCFSYIKLAVTLVKYFPQAYMNFYYKSTEGWSIGNVLLDFTGGSFSLLQMFLQSYNNDQWTLIFGDPTKFGLGVFSIVFDVVFFIQHFCLYRKRPGLQAARTGSGSRLRQDWAPSLQPKALPQTTSVSASSLKG

SEQ ID NO: 3 - DNA, 160 bp, Homo sapiens
cgcctctcccaaagtctagccgggcaggggaacgcggtgcattcctgaccggcacctggcgaggctcatgcgtcccgtgagggcggttcctcgagcctgggggcgctcaggtgagagcggacgcggcctcccctgtttcccaggcggaccccttgaggca

SEQ ID NO: 4 - DNA, 310 bp, Homo sapiens
ccttaccttctgctcagttgccgcctgggtctcggttggggaatttgcagattgctttggagacgctgagagaacctttgcgagagcgccggttgacgtgcggagtgcggggctccgggggactgagcagcacgagaccccatcctcccctccgggttttcacactgggcgaagggaggactcctgagctctgcctcttccagtaacattgaggattactgtgttttgtgagagctcgctaggcgccctaagcaacagaggtaaccactttatatccttggttctcaacctcgttattcctacctacccc

SEQ ID NO: 5 - DNA, 180 bp, Homo sapiens
agcagattcaacattcccctgaacttctctcttgctgtttttcttcctagttctgagaaatcgagaaacatgataaggaattggctgactatttttatcctttttcccctgaagctcgtagagaaatgtggtaagtttagaaaagacacgtcaactttgtaaagagggaaatggtggcta

SEQ ID NO: 6 - DNA, 179 bp, Homo sapiens
  misc_feature (7)..(7): n is a, c, g, or t
ggcctgnactctgacccagtgcctcatgtcattgatttgggtccttccagagtcaagcgtcagcctcactgttcctcctgtcgtaaagctggagaacggcagctcgaccaacgtcagcctcaccctgcggtaagttcctgggcctggcgctgtgctcagctccgctcaggccccgcagc

SEQ ID NO: 7 - DNA, 185 bp, Homo sapiens
gatctcactgtccagcttctcagcagtaattagactcttgtcctccacaggccaccattaaatgcaaccctggtgatcacttttgaaatcacatttcgttccaaaaatattactatccttgagctccccgatgaagtaagtaaccaatcttaacggatgggtagggaaatgctaggtaacaaaac

SEQ ID NO: 8 - DNA, 204 bp, Homo sapiens
tcctcggtaactgtacgtggcatcggattgaacctcagtcttcctaacaggttgtggtgcctcctggagtgacaaactcctcttttcaagtgacatctcaaaatgttggacaacttactgtttatctacatggaaatcactccaatcagaccgggtaggctggcctcagggtgtgggggcctcacgtgacaagaaggggcccgt

SEQ ID NO: 9 - DNA, 232 bp, Homo sapiens
ccttcataagcccagcctcagctcatcccggtccccaaactcctttccagcccgaggatacgctttcttgtgatccgcagcagcgccattagcatcataaaccaggtgattggctggatctactttgtggcctggtccatctccttctaccctcaggtgatcatgaattggaggcggaaaaggtaaccccctgggccgtatgtgcaggctctctcggggcccctaggagcag

SEQ ID NO: 10 - DNA, 200 bp, Homo sapiens
ccctgccagtcttcaccccctgccctgtcttgtccctccaccccctgcagtgtcattggtctgagcttcgacttcgtggctctgaacctgacaggcttcgtggcctacagtgtattcaacatcggcctcctctgggtgccctacatcaaggtacggccttgcctgccctacatctctgcccacatggcgtggtggcccgg

SEQ ID NO: 11 - DNA, 220 bp, Homo sapiens
tggtggcccggctgcccctcaccacccagcttctcccacccaccaaacaggagcagtttctcctcaaataccccaacggagtgaaccccgtgaacagcaacgacgtcttcttcagcctgcacgcggttgtcctcacgctgatcatcatcgtgcagtgctgcctgtatgaggtgagaccagccctggccccccacaggccaccccagccaacacccgccac

SEQ ID NO: 12 - DNA, 271 bp, Homo sapiens
cggcgtggcctctgtgtgggtccacatctctgccctcctctcgcccccagcgcggtggccagcgcgtgtcctggcctgccatcggcttcctggtgctcgcgtggctcttcgcatttgtcaccatgatcgtggctgcagtgggagtgatcacgtggctgcagtttctcttctgcttctcctacatcaagctcgcagtcacgctggtcaagtattttccacaggtacctccagggccctgttcacatggccggtggcaggagaggtgagagct

SEQ ID NO: 13 - DNA, 218 bp, Homo sapiens
ccgcccagccctcaccgccctccgtctgtatgtccgtctgtctggcccaggcctacatgaacttttactacaaaagcactgagggctggagcattggcaacgtgctcctggacttcaccgggggcagcttcagcctcctgcagatgttcctccagtcctacaacaacggtgagtcagccagcgggctgctggccaccctgcggctggggcatcgggcg

(19) United States
(12) Patent Application Publication
(10) Pub. No.: US 2022/0177918 A1
(43) Pub. Date: Jun. 9, 2022

(54) METHODS FOR TREATING INHERITED EYE DEFECTS

(71) Applicant: The Regents of the University of California, Oakland, CA (US)

(72) Inventor: Stephanie Cherqui, La Jolla, CA (US)

(21) Appl. No.: 17/602,987

(22) PCT Filed: Apr. 7, 2020
(86) PCT No.: PCT/US2020/027050

§ 371 Date: Oct. 11, 2021

Related U.S. Application Data
(60) Provisional application No. 62/833,422, filed on Apr. 12, 2019.

(51) Int. Cl. C12N 15/86; C07K 14/47; A61P 27/02; A61K 35/761

(52) U.S. Cl. CPC C12N 15/86; A61K 35/761; A61P 27/02; C07K 14/4702

(57) ABSTRACT

Provided herein are methods for treating an inherited eye disease or disorder through ex vivo introduction of a nucleic acid molecule into hematopoietic stem and progenitor cells (HSPCs) followed by transplantation of the HSPCs into a subject's eyes in need of treatment. Also provided are vectors containing the nucleic acid molecule.

### CROSS REFERENCE TO RELATED APPLICATION(S)

This application claims the benefit of priority under 35 U.S.C. §119(e) of U.S. Ser. No. 62/833,422, filed Apr. 12, 2019, the entire content of which is incorporated herein by reference.

### SEQUENCE LISTING

The instant application contains a Sequence Listing which has been submitted electronically in ASCII format and is hereby incorporated by reference in its entirety. Said ASCII copy, created on Apr. 6, 2020, is named 20378-202488_SL.txt and is 11 kilobytes in size.

### BACKGROUND OF THE INVENTION

### Field of the Invention

The invention relates generally to hereditary eye diseases and more specifically to treatment of such diseases with hematopoietic stem and progenitor cell (HSPC) gene therapy.

### Background Information

Cystinosis is characterized by the abnormal accumulation of the amino acid cystine in all cells of the body leading to multi-organ failure. Cystinosis is caused by mutations in the CTNS gene that codes for cystinosin, the lysosomal membrane-specific transporter for cystine. Intracellular metabolism of cystine, as it happens with all amino acids, requires its transport across the cell membrane. After degradation of endocytosed protein to cystine within lysosomes, it is normally transported to the cytosol. But if there is a defect in the carrier protein, cystine is accumulated in lysosomes. As cystine is highly insoluble, when its concentration in tissue lysosomes increase, its solubility is immediately exceeded, and crystalline precipitates are formed in almost all organs and tissues. In the eyes, crystals accumulate in the cornea causing photophobia and eventually blindness.

There are over 350 hereditary eye diseases, including (among others) albinism, aniridia, colorblindness, corneal dystrophies, glaucoma, keratoconus, Leber congenital amaurosis, night blindness, retinitis pigmentosa and retinoblastoma. Over sixty percent of infant blindness cases are inherited, including congenital cataracts, congenital glaucoma, retinal degeneration, optic atrophy and eye malformations. Ocular cystinosis is a benign, adult cystinosis form, which manifests as an accumulation of cystine crystals in the cornea and conjunctiva that results in tearing and photophobia. To date, there are no known cures or preventative measures for such hereditary eye diseases, with current therapies being directed to treating the associated symptoms. Thus, there is a need in the art for alternative or improved methods for treating hereditary eye diseases/disorders.

### SUMMARY OF THE INVENTION

Accordingly, in one aspect, the invention provides a method of treating an inherited eye disease or disorder in a subject. The method includes introducing a corresponding functional human protein associated with the inherited eye disease or disorder into hematopoietic stem and progenitor cells (HSPCs) of the subject, and transplanting the HSPCs into the subject, thereby treating the inherited eye disease or disorder. In various embodiments, the step of transplanting includes intracameral or intravitreal injection of the HSPCs into the subject's eye. Thus, when the inherited eye disease or disorder is ocular cystinosis, the corresponding functional human protein is cystinosin (CTNS).

In various embodiments, the step of introducing may include contacting a vector comprising a polynucleotide encoding the functional human protein associated with the inherited eye disease or disorder and a functional promoter with the HSPCs and allowing expression of the functional human protein associated with the inherited eye disease or disorder. In various embodiments, the inherited eye disease or disorder is ocular cystinosis and the functional human protein is CTNS. The subject may be a mammal, such as a human. In various embodiments, the vector is a viral vector selected from the group consisting of a lentiviral, adenoviral, and AAV vector. In various embodiments, the vector is a lentiviral vector. In various embodiments, the vector is an adenoviral vector. In various embodiments, the vector is an AAV vector. In various embodiments, the vector is a self-inactivating (SIN)-lentivirus vector, such as pCCL-CTNS. In various embodiments, the step of introducing is performed ex vivo. In various embodiments, the HSPCs are isolated from the blood or bone marrow of the subject.

In another aspect, the present invention provides a method of treating or ameliorating an inherited eye disease or disorder in a subject. The method includes isolating hematopoietic stem and HSPCs cells from a subject's blood or bone marrow, introducing a functional human gene into the HSPCs, wherein the gene encodes a protein corresponding to the inherited eye disease or disorder, and transplanting the HSPCs back into the subject, thereby treating or ameliorating the inherited eye disease or disorder. In various embodiments, the step of transplanting includes intracameral or intravitreal injection of the HSPCs into the subject's eye. Thus, when the inherited eye disease or disorder is ocular cystinosis, the functional human gene is CTNS. In various embodiments, the HSPCs are CD34+ cells.

In various embodiments, the step of introducing the functional human CTNS gene into the HSPCs includes using a vector, such as a viral vector. In various embodiments, the vector is a viral vector selected from the group consisting of a lentiviral, adenoviral, and AAV vector. In various embodiments, the step of introducing the functional human CTNS gene into the HSPCs comprises using a vector. In various embodiments, the level of cystine in the eye of the subject is reduced following treatment.

The subject may be on cysteamine therapy prior to treatment. In various embodiments, cystine or cystine crystals are measured in the eye prior to and/or following treatment. In various embodiments, cystine crystals are measured using in vivo confocal microscopy. In various embodiments, cystine levels may be measured prior to, during and/or following treatment. In various embodiments, cystine levels are measured using biological samples obtained from the subject, such as blood samples.

In another aspect, the present invention provides a method of treating or ameliorating an inherited eye disease or disorder in a subject. The method includes producing a functional human gene associated with the inherited eye disease or disorder in the subject using a gene editing system. Thus, when the inherited eye disease or disorder is ocular cystinosis, the functional human gene is CTNS. In various embodiments, the gene editing system is selected from the group consisting of CRISPR/Cas, zinc finger nucleases, and transcription activator-life effector nucleases. In various embodiments, the step of producing comprises administering to the subject an effective amount of a vector comprising the gene editing system. In various embodiments, the step of producing comprises obtaining a sample of cells from the subject, transfecting the gene editing system into the sample of cells, and thereafter, transplanting the transfected cells into the subject. In various embodiments, the step of transplanting comprises intracameral or intravitreal injection of the transfected cells into the eye of the subject. In various embodiments, the sample of cells is selected from the group consisting of blood cells and HSPCs.

In another aspect, the present invention provides a method of treating or ameliorating an inherited eye disease or disorder in a subject. The method includes contacting cells expressing a defective protein associated with the inherited eye disease or disorder from the subject with a vector encoding a gene editing system that, when transfected into the cells, corrects a mutation of an endogenous gene encoding the defective protein, thereby treating the inherited eye disease or disorder. Thus, when the inherited eye disease or disorder is ocular cystinosis, the protein is cystinosin (CTNS).

In various embodiments, the gene editing system is selected from the group consisting of CRISPR/Cas, zinc finger nucleases, engineered meganucleases, ARCUS, and transcription activator-life effector nucleases. In various embodiments, the step of contacting comprises administering to the subject an effective amount of the vector. In various embodiments, the step of contacting comprises obtaining a sample of cells from the subject, transfecting the gene editing system into the sample of cells, and thereafter, transplanting the transfected cells into the subject. In various embodiments, the step of transplanting includes intracameral or intravitreal injection of the transfected cells into the subject's eye. In various embodiments, the sample of cells is selected from the group consisting of blood cells and HSPCs.

### BRIEF DESCRIPTION OF THE DRAWINGS

FIG. 1 is a graphical diagram showing cystine crystal quanitification in the eyes. Cystine crystal quantification in the eyes collected from: wildtype (WT) mice; wildtype DsRed (DS) mice; DsRed Ctns^{-/-} mice (CD; Controls non transplanted)=Negative Control; Ctns^{-/-} mice transplanted with GFP+ WT HSCs via tail vein injection (TVI)=systemic transplantation=Positive Control; Ctns^{-/-} mice transplanted with GFP+ Ctns^{-/-} HSCs via tail vein injection (TVI)=systemic transplantation=Negative Control; Ctns^{-/-} mice injected in the eye via intravitreal injection with GFP+ Ctns HSCs; Ctns^{-/-} mice injected in the eye via intravitreal injection with GFP+ WT HSCs; Ctns^{-/-} mice injected in the eye via intracameral injection with GFP+ Ctns HSCs; and Ctns^{-/-} mice injected in the eye via intracameral injection with GFP+ WT HSCs. Significant cystine crystal decrease was observed in the eyes injected via intracameral injection of GFP+ WT HSCs compared to intracameral injection of GFP+ Ctns HSCs.

FIG. 2 is a graphical diagram showing cystine content measurement in the eyes collected from: wildtype (WT) mice; Ctns^{-/-} mice (Controls non transplanted)=Negative Control; Ctns^{-/-} mice transplanted with GFP+ Ctns^{-/-} HSCs via tail vein injection (TVI)=systemic transplantation=Negative Control; Ctns^{-/-} mice transplanted with GFP+ WT HSCs via tail vein injection (TVI)=systemic transplantation=Positive Control; Ctns^{-/-} mice injected in the eye via intravitreal injection with GFP+ Ctns HSCs; Ctns^{-/-} mice injected in the eye via intravitreal injection with GFP+ WT HSCs; Ctns^{-/-} mice injected in the eye via intracameral injection with GFP+ Ctns HSCs; and Ctns^{-/-} mice injected in the eye via intracameral injection with GFP+ WT HSCs. While the number of mice is low a trend of cystine decrease is observed in the eyes injected via intracameral injection of GFP+ WT HSCs compared to intracameral injection of GFP+ Ctns HSCs.

FIGS. 3A-3G show tables providing geographical distribution of reported mutations in the CTNS gene.

### DETAILED DESCRIPTION OF THE INVENTION

The present invention is based, in part, on the finding that a self-inactivating (SIN)-lentivirus vector containing human cystinosin (CTNS) cDNA and a functional promoter can be used to ex vivo gene-corrected patients' autologous hematopoietic stem and progenitor cells (HSPCs), which can then be re-transplanted into the eyes of patients. As a result, autologous transplant of of ex vivo corrected HSPCs can serve as a long-term source of providing missing proteins in the eye without presenting risks of immune response. While autologous HSPCs are used in the illustrative examples herein, one of skill in the art would recognize that other HSPCs would be useful as well (e.g., allogeneic).

Before the present compositions and methods are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

The term “comprising,” which is used interchangeably with “including,” “containing,” or “characterized by,” is inclusive or open-ended language and does not exclude additional, unrecited elements or method steps. The phrase “consisting of” excludes any element, step, or ingredient not specified in the claim. The phrase “consisting essentially of” limits the scope of a claim to the specified materials or steps and those that do not materially affect the basic and novel characteristics of the claimed invention. The present disclosure contemplates embodiments of the invention compositions and methods corresponding to the scope of each of these phrases. Thus, a composition or method comprising recited elements or steps contemplates particular embodiments in which the composition or method consists essentially of or consists of those elements or steps.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described.

The term “subject” or “host organism,” as used herein, refers to any individual or patient to which the subject methods are performed. Generally the subject is human, although as will be appreciated by those in the art, the subject may be an animal. Thus other animals, including mammals such as rodents (including mice, rats, hamsters and guinea pigs), cats, dogs, rabbits, farm animals including cows, horses, goats, sheep, pigs, etc., and primates (including monkeys, chimpanzees, orangutans and gorillas) are included within the definition of subject.

The term “biological sample,” refers to any sample taken from a participant, including but not limited to cells, blood, tissue, skin, urine, etc., or hair.

As used herein, the terms “treating” and “treatment” refer to delaying the onset of, retarding or reversing the progress of, reducing the severity of, or alleviating or preventing either the disease or condition to which the term applies, or one or more symptoms of such disease or condition. The condition can include a predisposition to a disease or disorder. The effect of the administration of the composition to the subject (either treating and/or preventing) can be, but is not limited to, the cessation of one or more symptoms of the condition, a reduction or prevention of one or more symptoms of the condition, a reduction in the severity of the condition, the complete ablation of the condition, a stabilization or delay of the development or progression of a particular event or characteristic, or minimization of the chances that a particular event or characteristic will occur.

The term “mitigating” refers to reduction or elimination of one or more symptoms of that pathology or disease, and/or a reduction in the rate or delay of onset or severity of one or more symptoms of that pathology or disease, and/or the prevention of that pathology or disease. In various embodiments, the reduction or elimination of one or more symptoms of pathology or disease can include, e.g., a measurable and sustained reduction in the quantity of cystine (e.g., crystalline cystine) in the lysosomes of a cell in the patient, such as a cell in the eye.

As used herein, the terms “reduce” and “inhibit” are used together because it is recognized that, in some cases, a decrease can be reduced below the level of detection of a particular assay. As such, it may not always be clear whether the expression level or activity is “reduced” below a level of detection of an assay, or is completely “inhibited.” Nevertheless, it will be clearly determinable, following a treatment according to the present methods.

The term “therapeutically effective amount” or “effective amount” means the amount of a compound or pharmaceutical composition that elicits the biological or medical response of a tissue, system, animal or human that is being sought by the researcher, veterinarian, medical doctor or other clinician. Thus, the term “therapeutically effective amount” is used herein to denote any amount of a formulation that causes a substantial improvement in a disease condition when applied to the affected areas repeatedly over a period of time. The amount varies with the condition being treated, the stage of advancement of the condition, and the type and concentration of formulation applied. Appropriate amounts in any given instance will be readily apparent to those skilled in the art or capable of determination by routine experimentation. In the context of cystinosin, an example of a therapeutically effective amount of an agent, such as a population of hematopoietic stem cells transduced, gene-edited, or otherwise modified to express a human cystinosin transgene, is an amount sufficient to reduce the quantity of cystine (e.g., crystalline cystine) in the lysosomes of a cell in the patient, such as a cell in the eye.

A “therapeutic effect,” as used herein, encompasses a therapeutic benefit and/or a prophylactic benefit as described herein.

A “dosage” or “dose” are defined to include a specified size, frequency, or exposure level are included within the definition.

As used herein “pharmaceutically acceptable carrier” encompasses any of the standard pharmaceutical carriers, such as a phosphate buffered saline solution, water and emulsions such as an oil/water or water/oil emulsion, and various types of wetting agents.

The terms “administration” or “administering” are defined to include an act of providing a compound or pharmaceutical composition of the invention to a subject in need of treatment. The phrases “parenteral administration” and “administered parenterally” as used herein means modes of administration other than enteral and topical administration, usually orally or by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal and infrasternal injection and infusion. The phrases “systemic administration,” “administered systemically,” “peripheral administration” and “administered peripherally” as used herein mean the administration of a compound, drug or other material other than directly into the central nervous system, such that it enters the subject's system and, thus, is subject to metabolism and other like processes. In various embodiments, the compound or pharmaceutical composition of the invention is administered via intracameral injection or intravitreal injection into an eye of the subject.

The terms “polypeptide,” “peptide,” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

The term “defective protein” refers to a protein that is structurally abnormal as compared to its wildtype and therefore disrupts the function of cells, tissues and/or organs of the body. Often defective proteins fail to fold into their normal configuration; in this misfolded state, the proteins can become toxic in some way (a gain of toxic function) or they can lose their normal function. As described herein, a mutation in a gene may lead to expression of a defective protein, resulting in a disease or disorder or a predisposition for a disease or disorder in the subject.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, α-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The term “polynucleotide” refers to a single- or double-stranded polymer of deoxyribonucleotide or ribonucleotide bases read from the 5′ to the 3′ end. Polynucleotides include RNA and DNA, and may be isolated from natural sources, synthesized in vitro, or prepared from a combination of natural and synthetic molecules. Sizes of polynucleotides are expressed as base pairs (abbreviated “bp”), nucleotides (“nt”), or kilobases (“kb”). Where the context allows, the latter two terms may describe polynucleotides that are single-stranded or double-stranded. When the term is applied to double-stranded molecules it is used to denote overall length and will be understood to be equivalent to the term “base pairs”. It will be recognized by those skilled in the art that the two strands of a double-stranded polynucleotide may differ slightly in length and that the ends thereof may be staggered as a result of enzymatic cleavage; thus all nucleotides within a double-stranded polynucleotide molecule may not be paired.

As used herein, the term “gene” means the deoxyribonucleotide sequences comprising the coding region of a structural gene. A “gene” may also include non-translated sequences located adjacent to the coding region on both the 5′ and 3′ ends such that the gene corresponds to the length of the full-length mRNA. The sequences which are located 5′ of the coding region and which are present on the mRNA are referred to as 5′ non-translated sequences. The sequences which are located 3′ or downstream of the coding region and which are present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene which are transcribed into heterogenous nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

As used herein, a “regulatory gene” or “regulatory sequence” is a nucleic acid sequence that encodes products (e.g., transcription factors) that control the expression of other genes.

The terms “gene transfer” or “gene delivery” refer to methods or systems for reliably inserting foreign DNA into host cells. Such methods can result in transient expression of non-integrated transferred DNA, extrachromosomal replication and expression of transferred replicons (e.g. episomes), or integration of transferred genetic material into the genomic DNA of host cells.

As used herein, a “vector” is a tool that allows or facilitates the transfer of an entity from one environment to another. It is a plasmid, phage, or cosmid, into which another DNA segment may be inserted so as to bring about the replication of the inserted segment in the appropriate prokaryotic or eukaryotic cell. Generally, a vector is capable of replication when associated with the proper control elements. In general, the term “vector” refers to a nucleic acid molecule capable of transporting another nucleic acid to which it has been linked. Vectors include, but are not limited to, nucleic acid molecules that are single-stranded, double-stranded, or partially double-stranded; nucleic acid molecules that comprise one or more free ends, no free ends (e.g. circular); nucleic acid molecules that comprise DNA, RNA, or both; and other varieties of polynucleotides known in the art.

The term “viral vector” refers to a vector wherein virally-derived polynucleotide sequences are present in the vector for transfection into a host cell. Thus, viral vectors can be particularly useful for introducing a polynucleotide useful in performing a method of the invention into a target cell. Viral vectors have been developed for use in particular host systems, particularly mammalian systems and include, for example, retroviral vectors, other lentivirus vectors such as those based on the human immunodeficiency virus (HIV), adenovirus vectors (AV), adeno-associated virus vectors (AAV), herpes virus vectors, vaccinia virus vectors, and the like (see Miller and Rosman, BioTechniques 7:980-990, 1992; Anderson et al., Nature 392:25-30 Suppl., 1998; Verma and Somia, Nature 389:239-242, 1997; Wilson, New Engl. I Med. 334:1185-1187 (1996), each of which is incorporated herein by reference). Lentivirus vectors have been most commonly used to achieve chromosomal integration.

An adeno-associated virus (AAV) is a small replication-defective, nonenveloped virus that depends on the presence of a second virus, such as adenovirus or herpes virus, for its growth in cells. Thus, the term “AAV vector” refers to a vector derived from an adeno-associated virus serotype, including without limitation, AAV1, AAV2, AAV3, AAV4, AAVS, AAV6, etc. In various embodiments, the AAV is an AAV9 particle. AAV vectors can have one or more of the AAV wild-type genes deleted in whole or part, e.g., the rep and/or cap genes, but retain functional flanking inverted terminal repeat (ITR) sequences. Functional ITR sequences are necessary for the rescue, replication and packaging of the AAV virion. Thus, an AAV vector is defined herein to include at least those sequences required in cis for replication and packaging (e.g., functional ITRs) of the virus. The ITRs need not be the wild-type nucleotide sequences, and may be altered, e.g., by the insertion, deletion or substitution of nucleotides, as long as the sequences provide for functional rescue, replication and packaging. AAV expression vectors are constructed using known techniques to at least provide as operatively linked components in the direction of transcription, control elements including a transcriptional initiation region, the DNA of interest (i.e., the TNNI3 gene) and a transcriptional termination region.

Additional references describing AAV vectors which could be used in the methods of the present invention include the following: Carter, B., Handbook of Parvoviruses, vol. I, pp. 169-228, 1990; Berns, Virology, pp. 1743-1764 (Raven Press 1990); Carter, B., Curr. Opin. Biotechnol., 3: 533-539, 1992; Muzyczka, N., Current Topics in Microbiology and Immunology, 158: 92-129, 1992; Flotte, T. R., et al., Am. I Respir. Cell Mol. Biol. 7:349-356, 1992; Chatterjee et al., Ann. NY Acad. Sci., 770: 79-90, 1995; Flotte, T. R., et al., WO 95/13365 (18 May 1995); Trempe, J. P., et al., WO 95/13392 (18 May 1995); Kotin, R., Human Gene Therapy, 5: 793-801, 1994; Flotte, T. R., et al., Gene Therapy 2:357-362, 1995; Allen, J. M., WO 96/17947 (13 Jun. 1996); and Du et al., Gene Therapy 3: 254-261, 1996. See also, U.S. Pat. No. 8,865,881, incorporated herein by reference.

If a viral vector specific for the cell type is not available, the vector can be modified to express a receptor (or ligand) specific for a ligand (or receptor) expressed on the target cell, or can be encapsulated within a liposome, which also can be modified to include such a ligand (or receptor). A peptide agent can be introduced into a cell by various methods, including, for example, by engineering the peptide to contain a protein transduction domain such as the human immunodeficiency virus TAT protein transduction domain, which can facilitate translocation of the peptide into the cell. In addition, there are a variety of biomaterial-based technologies such as nano-cages and pharmacological delivery wafers (such as used in brain cancer chemotherapeutics) which may also be modified to accommodate this technology.

As used herein, a “protein coding sequence” or a sequence that encodes a particular protein or polypeptide, is a nucleic acid sequence that is transcribed into mRNA (in the case of DNA) and is translated (in the case of mRNA) into a polypeptide in vitro or in vivo when placed under the control of appropriate regulatory sequences. The boundaries of the coding sequence are determined by a start codon at the 5′ terminus (N-terminus) and a translation stop nonsense codon at the 3′ terminus (C-terminus). A coding sequence can include, but is not limited to, cDNA from eukaryotic mRNA, genomic DNA sequences from eukaryotic DNA, and synthetic nucleic acids. A transcription termination sequence will usually be located 3′ to the coding sequence.

As used herein, a “promoter” is defined as a regulatory DNA sequence generally located upstream of a gene that mediates the initiation of transcription by directing RNA polymerase to bind to DNA and initiating RNA synthesis. A promoter can be a constitutively active promoter (i.e., a promoter that is constitutively in an active/“ON” state), it may be an inducible promoter (i.e., a promoter whose state, active/“ON” or inactive/“OFF”, is controlled by an external stimulus, e.g., the presence of a particular compound or protein), it may be a spatially restricted promoter (i.e., transcriptional control element, enhancer, etc.)(e.g., tissue specific promoter, cell type specific promoter, etc.), and it may be a temporally restricted promoter (i.e., the promoter is in the “ON” state or “OFF” state during specific stages of embryonic development or during specific stages of a biological process. Thus, in various embodiments, the promoter may be a stem cell-specific promoter that drives transgene expression. For example, constitutive promoters of different strengths can be used. Expression vectors and plasmids in accordance with the present invention may include one or more constitutive promoters, such as viral promoters or promoters from mammalian genes that are generally active in promoting transcription. Exemplary promoters include, but are not limited to, human Elongation Factor 1 alpha promoter (EFS), SV40 early promoter, mouse mammary tumor virus long terminal repeat (LTR) promoter; adenovirus major late promoter (Ad MLP); a herpes simplex virus (HSV) promoter, an endogenous cellular promoter that is heterologous to the gene of interest, a cytomegalovirus (CMV) promoter such as the CMV immediate early promoter region (CMVIE), a Rous sarcoma virus (RSV) promoter, synthetic promoters, hybrid promoters, and the like.

As used herein, an “enhancer” is a short (50-1500 bp) region of DNA that can be bound by proteins (activators) to increase the likelihood that transcription of a particular gene will occur. Thus, an enhancer may be used to increase promoter strength with regard to expression of the open reading frame for gene expression.

As used herein, the terms “functionally linked” and “operably linked” are used interchangeably and refer to a functional relationship between two or more DNA segments, in particular gene sequences to be expressed and those sequences controlling their expression. For example, a promoter/enhancer sequence, including any combination of cis-acting transcriptional control elements is operably linked to a coding sequence if it stimulates or modulates the transcription of the coding sequence in an appropriate host cell or other expression system. Promoter regulatory sequences that are operably linked to the transcribed gene sequence are physically contiguous to the transcribed sequence.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

As used herein, the term “genetic modification” is used to refer to any manipulation of an organism's genetic material in a way that does not occur under natural conditions. Methods of performing such manipulations are known to those of ordinary skill in the art and include, but are not limited to, techniques that make use of vectors for transforming cells with a nucleic acid sequence of interest. Included in the definition are various forms of gene editing in which DNA is inserted, deleted or replaced in the genome of a living organism using engineered nucleases, or “molecular scissors.” These nucleases create site-specific double-strand breaks (DSBs) at desired locations in the genome. The induced double-strand breaks are repaired through nonhomologous end-joining (NHEJ) or homologous recombination (HR), resulting in targeted mutations (i.e., edits).

There are several families of engineered nucleases used in gene editing, for example, but not limited to, meganucleases, zinc finger nucleases (ZFNs), transcription activator-like effector-based nucleases (TALEN), the CRISPR-Cas system, and ARCUS. However, it should be understood that any known gene editing system utilizing engineered nucleases may be used in the methods described herein.

CRISPR (Clustered Regularly Interspaced Short Palindromic Repeats) is an acronym for DNA loci that contain multiple, short, direct repetitions of base sequences. The prokaryotic CRISPR/Cas system has been adapted for use as gene editing (silencing, enhancing or changing specific genes) for use in eukaryotes (see, for example, Cong, Science, 15:339(6121):819-823 (2013) and Jinek, et al., Science, 337(6096):816-21 (2012)). By transfecting a cell with elements including a Cas gene and specifically designed CRISPRs, nucleic acid sequences can be cut and modified at any desired location. Methods of preparing compositions for use in genome editing using the CRISPR/Cas systems are described in detail in US Pub. No. 2016/0340661, US Pub. No. 20160340662, US Pub. No. 2016/0354487, US Pub. No. 2016/0355796, US Pub. No. 20160355797, and WO 2014/018423, which are specifically incorporated by reference herein in their entireties.

Thus, as used herein, “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g., tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer”, “guide RNA” or “gRNA” in the context of an endogenous CRISPR system), or other sequences and transcripts from a CRISPR locus. One or more tracr mate sequences operably linked to a guide sequence (e.g., direct repeat-spacer-direct repeat) can also be referred to as “pre-crRNA” (pre-CRISPR RNA) before processing or crRNA after processing by a nuclease.

In some embodiments, a tracrRNA and crRNA are linked and form a chimeric crRNA-tracrRNA hybrid where a mature crRNA is fused to a partial tracrRNA via a synthetic stem loop to mimic the natural crRNA:tracrRNA duplex as described in Cong, Science, 15:339(6121):819-823 (2013) and Jinek, et al., Science, 337(6096):816-21 (2012)). A single fused crRNA-tracrRNA construct can also be referred to as a guide RNA or gRNA (or single-guide RNA (sgRNA)). Within an sgRNA, the crRNA portion can be identified as the ‘target sequence’ and the tracrRNA is often referred to as the ‘scaffold’.

There are many resources available for helping practitioners determine suitable target sites once a desired DNA target sequence is identified. For example, numerous public resources, including a bioinformatically generated list of about 190,000 potential sgRNAs, targeting more than 40% of human exons, are available to aid practitioners in selecting target sites and designing the associate sgRNA to affect a nick or double strand break at the site. See also, crispr.u-psud.fr, a tool designed to help scientists find CRISPR targeting sites in a wide range of species and generate the appropriate crRNA sequences.

In some embodiments, one or more vectors driving expression of one or more elements of a CRISPR system are introduced into a target cell such that expression of the elements of the CRISPR system direct formation of a CRISPR complex at one or more target sites. While the specifics can be varied in different engineered CRISPR systems, the overall methodology is similar. A practitioner interested in using CRISPR technology to target a DNA sequence can insert a short DNA fragment containing the target sequence into a guide RNA expression plasmid. The sgRNA expression plasmid contains the target sequence (about 20 nucleotides), a form of the tracrRNA sequence (the scaffold) as well as a suitable promoter and necessary elements for proper processing in eukaryotic cells. Such vectors are commercially available (see, for example, Addgene). Many of the systems rely on custom, complementary oligos that are annealed to form a double stranded DNA and then cloned into the sgRNA expression plasmid. Co-expression of the sgRNA and the appropriate Cas enzyme from the same or separate plasmids in transfected cells results in a single or double strand break (depending of the activity of the Cas enzyme) at the desired target site.

Zinc-finger nucleases (ZFNs) are artificial restriction enzymes generated by fusing a zinc finger DNA-binding domain to a DNA-cleavage domain. Zinc finger domains can be engineered to target specific desired DNA sequences and this enables zinc-finger nucleases to target unique sequences within complex genomes. By taking advantage of endogenous DNA repair machinery, these reagents can be used to precisely alter the genomes of higher organisms. The most common cleavage domain is the Type IIS enzyme Fok1. Fok1 catalyzes double-stranded cleavage of DNA, at 9 nucleotides from its recognition site on one strand and 13 nucleotides from its recognition site on the other. See, for example, U.S. Pat. Nos. 5,356,802; 5,436,150 and 5,487,994; as well as Li et al. Proc., Natl. Acad. Sci. USA 89 (1992):4275-4279; Li et al. Proc. Natl. Acad. Sci. USA, 90:2764-2768 (1993); Kim et al. Proc. Natl. Acad. Sci. USA. 91:883-887 (1994a); Kim et al. J. Biol. Chem. 269:31,978-31,982 (1994b), all of which are incorporated herein by reference. One or more of these enzymes (or enzymatically functional fragments thereof) can be used as a source of cleavage domains.

Transcription activator-like effector nucleases (TALENs) have an overall architecture similar to that of ZFNs, with the main difference being that the DNA-binding domain comes from TAL effector proteins, transcription factors from plant pathogenic bacteria. The DNA-binding domain of a TALEN is a tandem array of amino acid repeats, each about 34 residues long. The repeats are very similar to each other; typically they differ principally at two positions (amino acids 12 and 13, called the repeat variable diresidue, or RVD). Each RVD specifies preferential binding to one of the four possible nucleotides, meaning that each TALEN repeat binds to a single base pair, though the NN RVD is known to bind adenines in addition to guanine. TAL effector DNA binding is mechanistically less well understood than that of zinc-finger proteins, but their seemingly simpler code could prove very beneficial for engineered-nuclease design. TALENs also cleave as dimers, have relatively long target sequences (the shortest reported so far binds 13 nucleotides per monomer) and appear to have less stringent requirements than ZFNs for the length of the spacer between binding sites. Monomeric and dimeric TALENs can include more than 10, more than 14, more than 20, or more than 24 repeats. Methods of engineering TAL to bind to specific nucleic acids are described in Cermak, et al, Nucl. Acids Res. 1-11 (2011); US Published Application No. 2011/0145940, which discloses TAL effectors and methods of using them to modify DNA; Miller et al. Nature Biotechnol 29: 143 (2011) reported making TALENs for site-specific nuclease architecture by linking TAL truncation variants to the catalytic domain of Fokl nuclease. The resulting TALENs were shown to induce gene modification in immortalized human cells. General design principles for TALE binding domains can be found in, for example, WO 2011/072246. Each of the foregoing references are incorporated herein by reference in their entireties.

The nuclease activity of the genome editing systems described herein cleave target DNA to produce single or double strand breaks in the target DNA. Double strand breaks can be repaired by the cell in one of two ways: non-homologous end joining, and homology-directed repair. In non-homologous end joining (NHEJ), the double-strand breaks are repaired by direct ligation of the break ends to one another. As such, no new nucleic acid material is inserted into the site, although some nucleic acid material may be lost, resulting in a deletion. In homology-directed repair, a donor polynucleotide with homology to the cleaved target DNA sequence is used as a template for repair of the cleaved target DNA sequence, resulting in the transfer of genetic information from a donor polynucleotide to the target DNA. As such, new nucleic acid material can be inserted/copied into the site. Therefore, in some embodiments, the genome editing vector or composition optionally includes a donor polynucleotide. The modifications of the target DNA due to NHEJ and/or homology-directed repair can be used to induce gene correction, gene replacement, gene tagging, transgene insertion, nucleotide deletion, gene disruption, gene mutation, etc.

Accordingly, cleavage of DNA by the genome editing vector or composition can be used to delete nucleic acid material from a target DNA sequence by cleaving the target DNA sequence and allowing the cell to repair the sequence in the absence of an exogenously provided donor polynucleotide. Alternatively, if the genome editing composition includes a donor polynucleotide sequence that includes at least a segment with homology to the target DNA sequence, the methods can be used to add, i.e., insert or replace, nucleic acid material to a target DNA sequence (e.g., to “knock in” a nucleic acid that encodes for a protein, an siRNA, an miRNA, etc.), to add a tag (e.g., 6xHis), a fluorescent protein (e.g., a green fluorescent protein; a yellow fluorescent protein, etc.), hemagglutinin (HA), FLAG, etc.), to add a regulatory sequence to a gene (e.g., promoter, polyadenylation signal, internal ribosome entry sequence (IRES), 2A peptide, start codon, stop codon, splice signal, localization signal, etc.), to modify a nucleic acid sequence (e.g., introduce a mutation), and the like. As such, the compositions can be used to modify DNA in a site-specific, i.e., “targeted” way, for example gene knock-out, gene knock-in, gene editing, gene tagging, etc., as used in, for example, gene therapy.

ARCUS is a genome editing platform derived from a natural genome editing enzyme referred to as a “homing endonuclease.” Homing endonucleases are site-specific DNA-cutting enzymes encoded in the genomes of many eukaryotic species that are able to precisely recognize long DNA sequences (12-40 base pairs). These non-destructive enzymes trigger gene conversion events that modify the genome in a very precise way, most frequently by the insertion of a new DNA sequence. Thus, the ARCUS genome editing platform relies upon engineered ARC nucleases, which are fully synthetic enzymes similar to a homing endonuclease, but with improved specificity to recognize a DNA sequence within any target gene.

The term “antibody” as used herein refers to polyclonal and monoclonal antibodies and fragments thereof, and immunologic binding equivalents thereof. The term “antibody” refers to a homogeneous molecular entity, or a mixture such as a polyclonal serum product made up of a plurality of different molecular entities, and broadly encompasses naturally-occurring forms of antibodies (for example, IgG, IgA, IgM, IgE) and recombinant antibodies such as single-chain antibodies, chimeric and humanized antibodies and multi-specific antibodies. The term “antibody” also refers to fragments and derivatives of all of the foregoing, and may further comprise any modified or derivatized variants thereof that retains the ability to specifically bind an epitope. Antibody derivatives may comprise a protein or chemical moiety conjugated to an antibody. A monoclonal antibody is capable of selectively binding to a target antigen or epitope. Antibodies may include, but are not limited to polyclonal antibodies, monoclonal antibodies (mAbs), humanized or chimeric antibodies, camelized antibodies, single chain antibodies (scFvs), Fab fragments, F(ab′)₂ fragments, disulfide-linked Fvs (sdFv) fragments, for example, as produced by a Fab expression library, anti-idiotypic (anti-Id) antibodies, intrabodies, nanobodies, synthetic antibodies, and epitope-binding fragments of any of the above.

As used herein, the term “stem cell” refers to undifferentiated or partically differentiated cells that can differentiate into various types of cells and divide indefinitely to produce more of the same stem cell. The term “progenitor cell” refers to a cell that can differentiate into a specific type of cell, but is already more specific than a stem cell and is pushed to differentiate into its “target” cell. Unlike a stem cell, a progenitor cell can divide only a limited number of times.

As used herein, “hematopoietic stem cells” (HSCs) refer to stem cells that give rise to other blood cells. HSCs possess the ability of multipotency (i.e., one HSC can differentiate into all functional blood cells) and self-renewal (i.e., HSCs can divide and give rise to an identical daughter cell, without differentiation). Through a series of lineage commitment steps, HSCs give rise to progeny that progressively lose self-renewal potential and successively become more and more restricted in their differentiation capacity, generating multi-potential and lineage-committed progenitor cells, and ultimately mature functional circulating blood cells.

The ability of hematopoietic stem and progenitor cells (HSPCs) to self-renew and differentiate is fundamental for the formation and maintenance of life-long hematopoiesis and deregulation of these processes may lead to severe clinical consequences. HSPCs are also highly valuable for their ability to reconstitute the hematopoietic system when transplanted and this has enabled their use in the clinic to treat a variety of disorders including bone marrow failure, myeloproliferative disorders and other acquired or genetic disorders that affect blood cells.

As used herein, a “pluripotent cell” refers to a cell derived from an embryo produced by activation of a cell containing DNA of all female or male origin that can be maintained in vitro for prolonged, theoretically indefinite period of time in an undifferentiated state that can give rise to different differentiated tissue types, i.e., ectoderm, mesoderm, and endoderm. “Embryonic stem cells” (ES cells) are pluripotent stem cells derived from the inner cell mass of a blastocyst, an early-stage preimplantation embryo.

As used herein, an “autologous transplant” refers to a transplant that uses a subject's own stem cells. These cells are collected in advance and returned at a later stage. An “allogeneic transplant” refers to a transplant where the donor and the recipient of the stem cells are different people. Exemplary allogeneic cells include, but are not limited to, syngeneic cells, MHC-matched cells, etc.

Cystinosis is an autosomal metabolic disease that belongs to the family of the lysosomal storage disorders. Cystinosis has a devastating impact on the affected individuals, primarily children and young adults, even with cysteamine treatment. The prevalence of cystinosis is 1:100,000 to 1:200,000. The gene involved in cystinosis is the CTNS gene that encodes for the 7-transmembrane lysosomal cystine transporter, cystinosin. The CTNS gene consists of 12 exons with exons 3-12 being coding, and CTNS mutations result in either complete absence or reduced cystine transporting function.

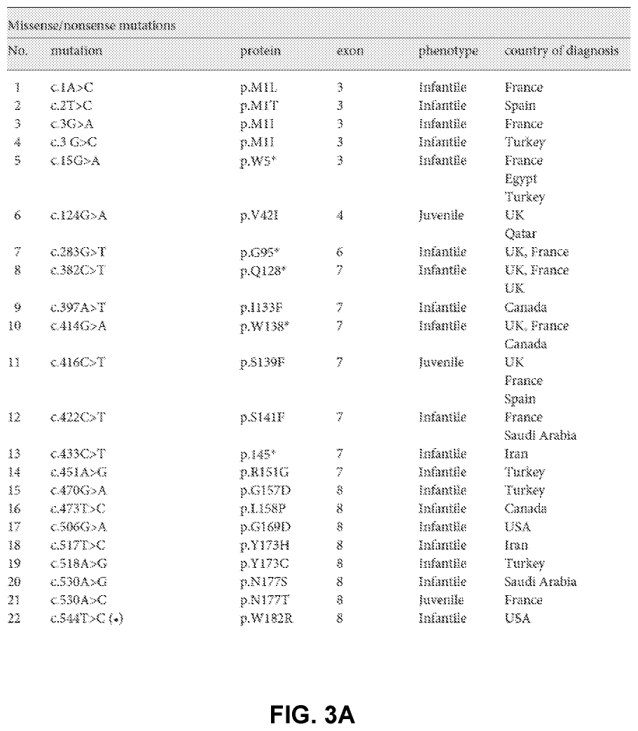

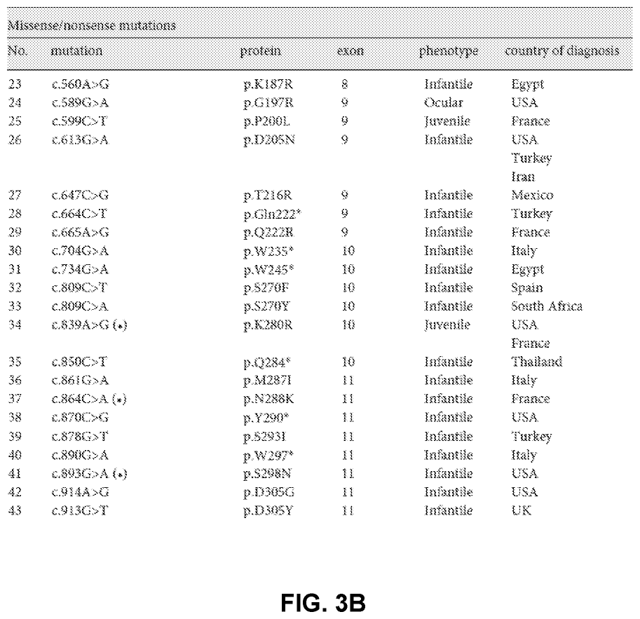

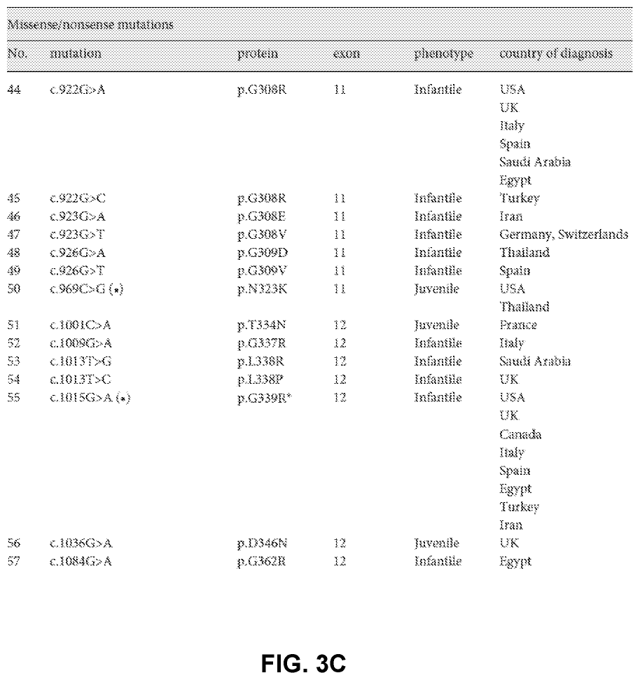

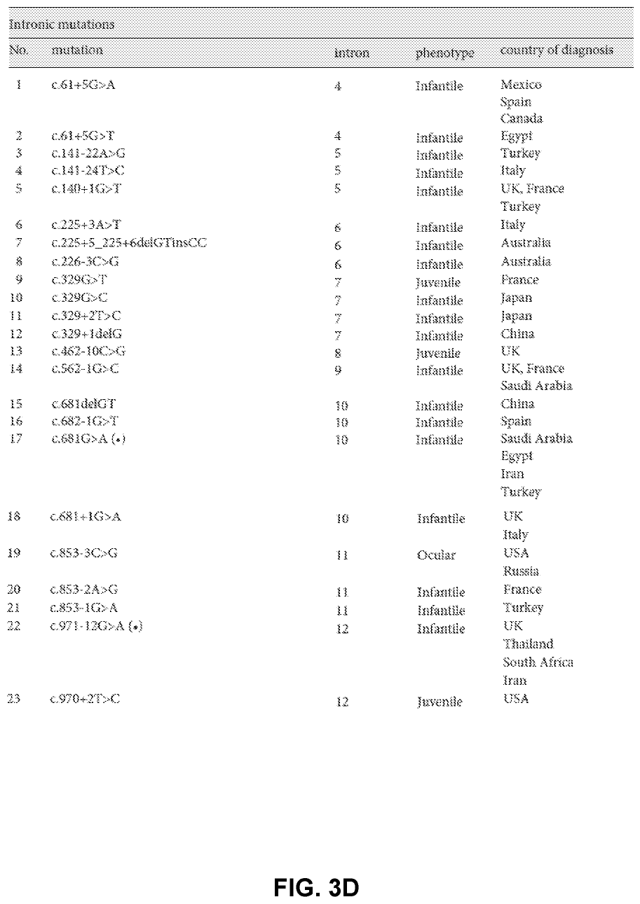

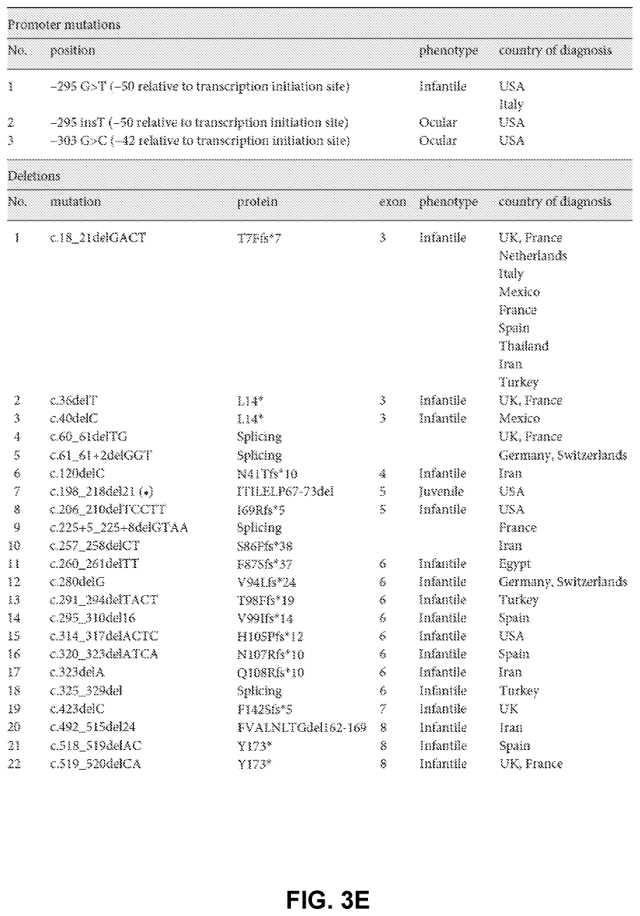

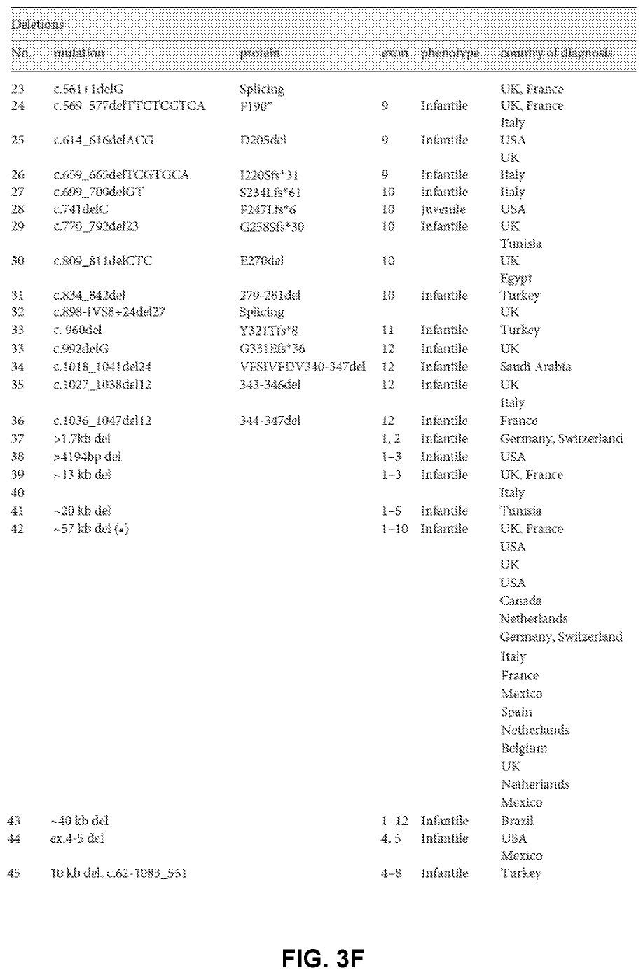

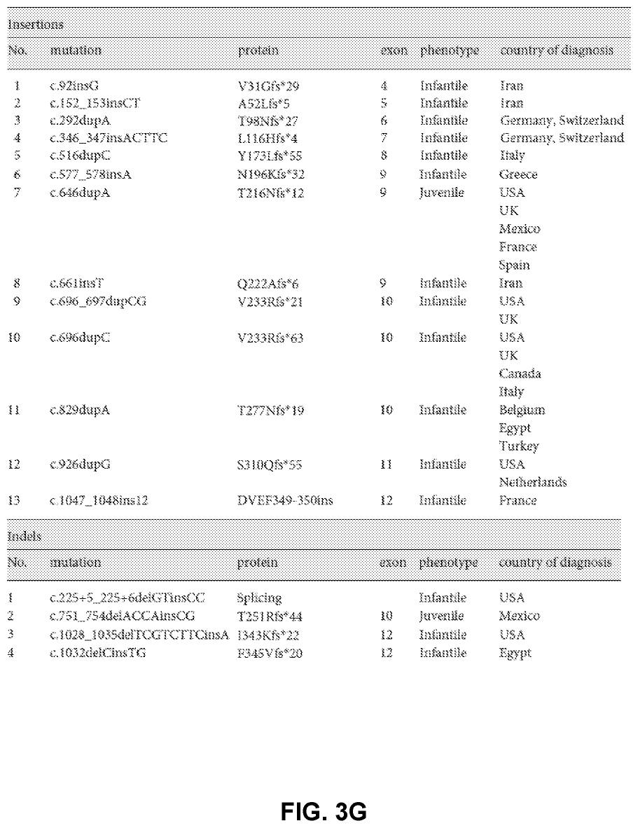

One of the most common mutations is a 57,257 base pair deletion commonly referred to as the 57 kb deletion, which includes exons 1-9 and part of exon 10 of CTNS. Over 140 different pathogenic CTNS mutations have been identified in diverse world populations, including 57 missense and nonsense mutations, 23 intronic mutations, 45 deletions, 13 small insertions, 4 indels and 3 promoter region mutations, as set forth in FIGS. 3A-3G (see, e.g., David et al. Molecular basis of cystinosis: geographic distribution, functional consequences of mutations in the CTNS gene, and potential for repair. Nephron. 2019;141(2):133-46; and Anikster, et al. Mol. Genet. Metab., 66 (1999), pp. 111-116). The type and extent of mutation determines the type and severity of cystinosis in the carrier (see, e.g., Attard et al. (December 1999). “Severity of phenotype in cystinosis varies with mutations in the CTNS gene: predicted effect on the model of cystinosin”. Human Molecular Genetics. 8 (13):2507-14). This is a result of the degree of transport inhibition caused by the misfolding of cystinosin. Each of the foregoing references are incorporated herein by reference in their entireties.

Cystinosis as a clinical entity is a progressive dysfunction of multiple organs caused by the accumulation of cystine in the lysosomes of all the cells in the body; affected patients store 50-100 times the normal amounts of cystine in their cells. Cystine storage leads to the formation of cystine crystals in all tissues. The main clinical complications in cystinosis include diabetes, hypothyroidism, myopathy and central nervous system deterioration. Corneal cystine crystals appear from the first decade of life resulting in photophobia and visual impairment.

The cystinosis phenotype is typically divided into three clinical forms. The most severe variant, infantile nephropathic cystinosis affects ˜95% of patients and is characterized by the development of renal Fanconi syndrome during the first months of life followed by glomerular dysfunction, which if untreated, results in end-stage kidney disease (ESKD) around the age of 10 years. Late-onset juvenile nephropathic type usually presents during childhood or at adolescence with mild or even absent proximal tubular dysfunction, proteinuria, which can be in the nephrotic range, and a slower rate of progression towards ESKD. Non-nephropathic cystinosis is a benign variant presenting with photophobia due to cystine accumulation in the cornea but causing no systemic organ damage.

The current treatment for cystinosis is the drug cysteamine (mercaptoethylamine), which reduces the intracellular cystine content. However, this therapy only delays disease progression and has no effect on renal Fanconi syndrome nor does it prevent end stage renal failure in affected patients. Cysteamine has also been shown to be inefficient to improve cellular dysfunctions in CTNS-deficient cells, proving that cellular defects in cystinosis are not only due to cystine accumulation but also due to the lack of the cystinosin itself that interacts directly with key cellular components.

In addition, cysteamine must be taken every 6 hours including at night, and results in bad body odor as well as severe gastrointestinal side effects such as vomiting and diarrhea that render treatment compliance difficult. In 2013, a delayed-release formulation of cysteamine (PROCYSBI®) was FDA-approved, which requires dosing every 12 hours. While PROCYSBI® reduces the number of doses improving the patients' quality of life, the impact on the disease is similar than immediate release cysteamine and patients still experience gastric side effects. Moreover, the cost of this medication is very high, $300,000-$600,000 per year per patient.

The ocular pathology in cystinosis requires topical administration of cysteamine eye drops every hour, which causes irritation and burning so compliance is very challenging. The cost of eye drops is about $50,000 per year per patient. Cysteamine and the supportive treatment for all the complications associated with cystinosis requires patients to take up to 60 pills per day; kids often require placement of a gastric tube to be able to tolerate the medications and get essential caloric intake. Medical complications increase in severity and number with age resulting in new and ever-increasing symptoms and treatments. There are unending doctor appointments, G-tube feedings, frequent blood draws, growth hormones shots, bone pain, daily vomiting, eye pain and severe gastrointestinal side effects. As the disease progress, their bodies deteriorate. The most severe complications for adults are myopathy, pulmonary issues and progression of corneal cystinosis. Patients with renal failure require dialysis or transplantation, both of which have significant negative health effects and due to the severe shortage of donor organs, patients may wait three to six years for transplantation. Thus, the current standard of care does not prevent the progression of the disease and significantly impacts the quality of life for patients with cystinosis who still die in early adulthood.

The inventors previously showed that systemic wildtype hematopoietic stem progenitor cell (HSPC) transplantation rescue the corneal defects in a mouse model of cystinosis (Ctns^{-/-} mice). Because there is an ocular non-nephropathic form of cystinosis, it was desirable to test if a local injection of HSPCs would have the same impact on the corneal defects. As such, intracameral and intravitreal HSPC injections were performed in the Ctns^{-/-} mice.

It was observed that systemic and intracameral injections led to significant reduction of the number corneal of crystals compared to controls. Thus, this shows that local injection of HSC can be a treatment for inherited eye defects. Accordingly, the invention provides use of ex vivo gene-modified HSPCs injected directly in the eye as a treatment of inherited eye defects.

Thus, the present disclosure demonstrates that one-time hematopoietic stem and progenitor cell (HSPC) transplantation holds the potential to become a long-term source of the missing protein cystinosin in inherited eye defects, diseases, and disorders. The therapy may further prevent blindness and long-term complications associated with cystinosis including unexpectedly the clearance of the corneal cystine crystals. This should also allow patients to withdraw from oral cysteamine, cysteamine eye drops and any other medications used for treating symptoms associated with the disease. As such, the quality of life of the patients is greatly improved and the cost of treatment highly decreased.

Due to the multi-systemic nature of cystinosis and all the drugs necessary to compensate for the absence of the protein, cystinosin, in every tissue, a gene therapy approach was investigated. Gene therapy has the potential to become an important new approach for the third millennium to treat both rare and common severe diseases because its reach extends well beyond that of conventional drugs and offers the prospect of a curative stem cell-based therapy with limited risks as compared to allogeneic HSC transplantation. Hematopoietic stem and progenitor cells (HSPCs) are therefore ideal candidates for use in regenerative medicine and cell replacement therapies because of their ease of isolation, self-renewal capacity, and safety. Moreover, gene therapy can address unmet medical need such as in the case of cystinosis, especially this strategy overcomes the unavailability of matched HSC donor and makes the treatment potentially available to all patients.

Using a rodent model of cystinosis (Ctns^{-/-} mice), it has been shown that transplantation of HSCs expressing a functional Ctns gene resulted in rescue of the corneal defects. It was found that HSPC transplantation led to substantial decreases in corneal cystine crystals, restored normal corneal thickness, and lowered intraocular pressure (TOP) in Ctns^{-/-} mice. It has also been demonstrated that HSPC-derived progeny differentiated into macrophages, which displayed tunneling nanotubes capable of transferring cystinosin-bearing lysosomes to diseased cells in the cornea.

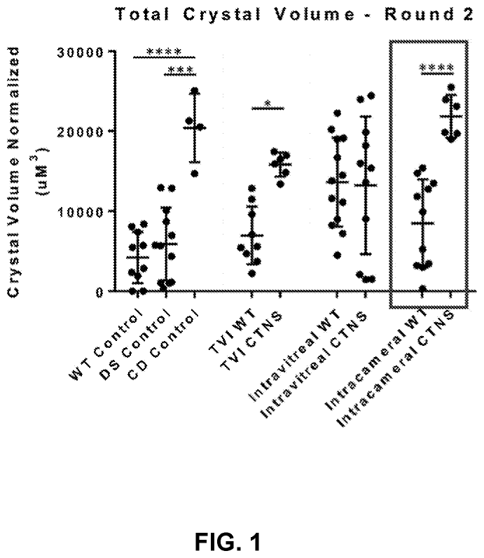

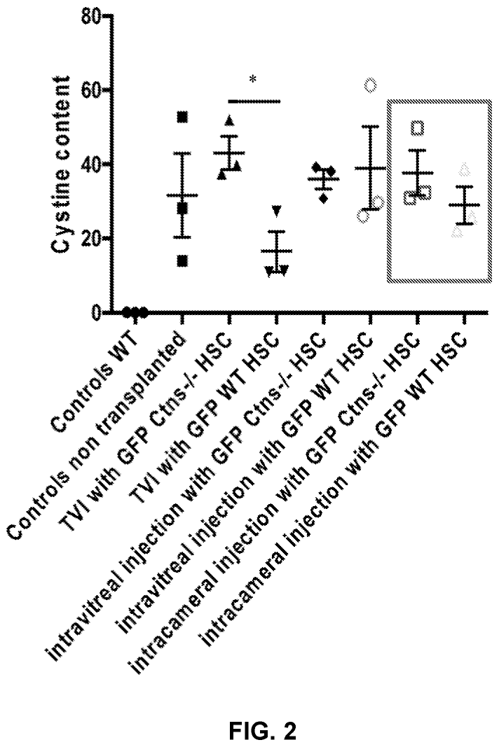

To determine if a local injection of HSPCs would have the same impact on the corneal defects, intracameral and intravitreal HSPC injections in 2-month-old animals were performed. The two groups were compared to mice systematically transplanted with HSPCs as well as non-transplanted control mice. At 6-months post transplantation all the mice were subjected to in vivo confocal microscopy to quantify corneal cystine crystals. It was found that systemic and intracameral injections led to significant reduction of the number of crystals compared to control (FIG. 1). These findings are confirmed by mass spectrometry with a reduction of cystine content in those 2 groups (FIG. 2).

The fate and phenotype of the HSPC progeny transplanted locally are now being investigated by confocal microscopy. Likewise, it will be determined whether they also differentiate in macrophages that are capable of providing functional cystinosin to the disease adjacent cells. This work is a proof of concept for ocular cystinosis, as well as other inherited eye disorders. Exemplary inherited eye diseases/disorders include, but are not limited to, albinism, aniridia, colorblindness, corneal dystrophies, glaucoma, keratoconus, Leber congenital amaurosis, night blindness, retinitis pigmentosa and retinoblastoma. Additionally, over sixty percent of infant blindness cases are inherited, including congenital cataracts, congenital glaucoma, retinal degeneration, optic atrophy and eye malformations.

As such, the present disclosure evaluates the impact of HSPC transplantation in a mouse model for cystinosis (Ctns^{-/-} mice). The present disclosure therefore demonstrates that transplantation of wildtype (WT) murine hematopoietic stem cells (mHSCs) led to a significant reduction of the number of crystals in the eye compared to controls. Given the risks of mortality and morbidity associated with allogeneic HSC transplantation, such as graft-versus-host diseases (GVHD), an autologous transplantation protocol of HSCs was developed for ex vivo modification. Using a self-inactivated-lentiviral vector (SIN-LV) to introduce a functional version of the CTNS cDNA, pCCL-CTNS (backbone pCCL-EFS-X-WPRE), efficacy in Ctns^{-/-} mice has been shown.

In vitro studies using human CD34⁺ HSPCs isolated from peripheral blood of healthy donors and cystinosis patients have now been completed, and the serial transplantation in the Ctns^{-/-} mice has been significantly advanced. Thus, efficacy of transplantation of CD34⁺ HSCs from G-CSF mobilized peripheral blood stem cells (PBSC) of patients with cystinosis, modified by ex vivo transduction using the pCCL-CTNS LV has been demonstrated.

Accordingly, in one aspect, the invention provides a method of treating an inherited eye disease or disorder in a subject. The method includes introducing ex vivo a functional human transmembrane protein or a nucleic acid molecule encoding a functional human transmembrane protein corresponding to the eye disease or disorder (e.g., cystinosis) to be treated into HSPCs of the subject, and thereafter transplanting the HSPCs into the subject, thereby treating the inherited eye disease or disorder. Thus, for example, when the disease or disorder to be treated is cystinosis, the functional human transmembrane protein to be introduced is cystinosoin (CTNS). In various embodiments, the nucleic acid molecule encoding CTNS may be delivered using a vector, such as a self-inactivating (SIN)-lentivirus vector, which may be, for example, pCCL-CTNS. In various embodiments, the step of introducing may include contacting a vector comprising a polynucleotide encoding the functional protein (e.g., CTNS) and a functional promoter (e.g., a ubiquitous or endogenous promoter of the fuctional protein) with the HSPCs and allowing expression of the functional protein. As such, the present disclosure provides a method for autologous transplantation of ex vivo gene-modified HSPCs to introduce a functional protein associated with a specific inherited eye disease or disorder.

Table 1 sets forth the exemplary inherited eye disease or disorder to be treated with ex vivo introduction of corresponding functional human transmembrane proteins.

TABLE 1

| | | | Protein size, # of |
|---|---|---|---|
| Human | Causative | Protein name | transmembrane |
| disease/disorder | gene, locus | (aliases) | helices (TM) |
| Ocular cystinosis | CTNS, 17p13 | Cystinosin | 367 aa; 7 TM |

Vectors derived from lentiviruses have supplanted γ-retroviral vector for gene therapy due to their superior gene transfer efficiency and better biosafety profile. Indeed, all cases of leukemogenic complications observed to date in clinical trials or animal models involved the use of retroviral vectors with LTR containing strong enhancer/promoters that can trigger distant enhancer activation. In contrast, the third-generation of lentivirus vectors, SIN-LV, with the deletions in their LTR, contains only one internal enhancer/promoter, which reduces the incidence of interactions with nearby cellular genes, and thus, decreases the risk of oncogenic integration. SIN-LV are also designed to prevent the possibility of developing replication competent lentivirus (RCL) during production of viral supernatants with three packaging plasmids necessary for production. Lentivirus vectors efficiently transduce HSPCs and do not alter their repopulation properties, which make this type of vector an attractive vehicle for stem cell gene therapy.

Clinical trials using SIN-LV to gene-correct human HSPCs are being undertaken in the U.S. and Europe for several conditions including HIV-1, β-thalassemia, immune deficiencies, metabolic diseases and cancers. For immune deficiency disorders, 35 patients have been transplanted with SIN-LV-modified HSPCs so far. A clinical trial in patients with Adrenoleukodystrophy (ALD) has achieved stable gene correction in ˜20% of hematopoietic cells in two patients. Cerebral demyelination was arrested without further progression over three years of follow-up, which represents a clinical outcome comparable to that observed after allogeneic transplantation; there was no evidence of clonal dominance. Recently, a clinical trial for Wilskott-Aldrich syndrome was reported in three patients 32 months post-transplantation. Stable and long-term engraftment of the gene-modified HSPCs (25-50%) resulted in improved platelet counts, protection from bleeding and infections, and resolution of eczema. Another clinical success was recently reported in three pre-symptomatic patients with Metachromatic Leukodystrophy. Transduced cell-derived blood cell engraftment achieved 45 to 80%, and up to 24 months later, protein activity was reconstituted to above normal values in cerebrospinal fluid associated with a clear therapeutic benefit.

The recent gene therapy successes using AAV vectors in the MCK mice not only prevented heart failure when given to presymptomatic animals, but also reversed the cardiomyopathy when given after the onset. While encouraging, this approach presents potential safety and logistic concerns: i) localized delivery by direct viral injection to affected sites poses certain challenges in accessing sites such as heart and brain and leads only to tissue-specific rescue, ii) systemic AAV delivery remains difficult in humans due to the high levels of vector necessary, leading to vector synthesis and safety concerns. In contrast, HSPC gene therapy approach has the key advantages: i) it treats all the complications by a single infusion of stem cells, ii) gene-correction occurs ex vivo in a controlled environment allowing cell characterization prior to transplantation, iii) it avoids immune reaction as compared to allogeneic transplantation. Thus, autologous HSPC gene therapy could provide a cure for inherited eye disease or disorder.

Amino acid and nucleic acid sequences for the human proteins set forth in Table 1 are known in the art. See, for example, human Cystinosin, Isoform 1, Accession No. 060931 (SEQ ID NO: 1):

MIRNWLTIFILFPLKLVEKCESSVSLTVPPVVKLENGSSTNVSLTLRPPL
NATLVITFEITFRSKNITILELPDEVVVPPGVTNSSFQVTSQNVGQLTVY
LHGNHSNQTGPRIRFLVIRSSAISIINQVIGWIYFVAWSISFYPQVIMNW
RRKSVIGLSFDFVALNLTGFVAYSVFNIGLLWVPYIKEQFLLKYPNGVNP
VNSNDVFFSLHAVVLTLIIIVQCCLYERGGQRVSWPAIGFLVLAWLFAFV
TMIVAAVGVITWLQFLFCFSYIKLAVTLVKYFPQAYMNFYYKSTEGWSIG
NVLLDFTGGSFSLLQMFLQSYNNDQWTLIFGDPTKFGLGVFSIVFDVVFF
IQHFCLYRKRPGYDQLN

human Cystinosin, Isoform 2, Accession No. 060931-2 (SEQ ID NO: 2):

MIRNWLTIFILFPLKLVEKCESSVSLTVPPVVKLENGSSTNVSLTLRPPL
NATLVITFEITFRSKNITILELPDEVVVPPGVTNSSFQVTSQNVGQLTVY
LHGNHSNQTGPRIRFLVIRSSAISIINQVIGWIYFVAWSISFYPQVIMNW
RRKSVIGLSFDFVALNLTGFVAYSVFNIGLLWVPYIKEQFLLKYPNGVNP
VNSNDVFFSLHAVVLTLIIIVQCCLYERGGQRVSWPAIGFLVLAWLFAFV
TMIVAAVGVTTWLQFLFCFSYIKLAVTLVKYFPQAYMNFYYKSTEGWSIG
NVLLDFTGGSFSLLQMFLQSYNNDQWTLIFGDPTKFGLGVFSIVFDVVFF
IQHFCLYRKRPGLQAARTGSGSRLRQDWAPSLQPKALPQTTSVSASSLKG

GenBank Accession No.: Y15922.1, human CTNS gene, exon 1 and 3′ flanking intronic region, which provides the nucleic acid sequence (SEQ ID NO: 3):

cgcctctccc aaagtctagc cgggcagggg aacgcggtgc
attcctgacc ggcacctggc gaggctcatg cgtcccgtga
gggcggttcc tcgagcctgg gggcgctcag gtgagagcgg
acgcggcctc ccctgtttcc caggcggacc ccttgaggca

GenBank Accession No.: Y15923.1, human CTNS gene, exon 2 and flanking intronic region, which provides the nucleic acid sequence (SEQ ID NO: 4):

ccttaccttc tgctcagttg ccgcctgggt ctcggttggg
gaatttgcag attgctttgg agacgctgag agaacctttg
cgagagcgcc ggttgacgtg cggagtgcgg ggctccgggg
gactgagcag cacgagaccc catcctcccc tccgggtttt
cacactgggc gaagggagga ctcctgagct ctgcctcttc
cagtaacatt gaggattact gtgttttgtg agagctcgct
aggcgcccta agcaacagag gtaaccactt tatatccttg
gttctcaacc tcgttattcc tacctacccc

GenBank Accession No.: Y15924.1, human CTNS gene, exon 3, flanking intronic regions and joined CDS, which provides the nucleic acid sequence (SEQ ID NO: 5):

agcagattca acattcccct gaacttctct cttgctgttt
ttcttcctag ttctgagaaa tcgagaaaca tgataaggaa
ttggctgact atttttatcc tttttcccct gaagctcgta
gagaaatgtg gtaagtttag aaaagacacg tcaactttgt
aaagagggaa atggtggcta

GenBank Accession No.: Y15925.1, human CTNS gene, exon 4 and flanking intronic regions, which provides the nucleic acid sequence (SEQ ID NO: 6):

ggcctgnact ctgacccagt gcctcatgtc attgatttgg
gtccttccag agtcaagcgt cagcctcact gttcctcctg
tcgtaaagct ggagaacggc agctcgacca acgtcagcct
caccctgcgg taagttcctg ggcctggcgc tgtgctcagc
tccgctcagg ccccgcagc

GenBank Accession No.: Y15926.1, human CTNS gene, exon 5 and flanking intronic regions, which provides the nucleic acid sequence (SEQ ID NO: 7):

gatctcactg tccagcttct cagcagtaat tagactcttg
tcctccacag gccaccatta aatgcaaccc tggtgatcac
ttttgaaatc acatttcgtt ccaaaaatat tactatcctt
gagctccccg atgaagtaag taaccaatct taacggatgg
gtagggaaat gctaggtaac aaaac

GenBank Accession No.: Y15927.1, human CTNS gene, exon 6 and flanking intronic regions, which provides the nucleic acid sequence (SEQ ID NO: 8):

tcctcggtaa ctgtacgtgg catcggattg aacctcagtc
ttcctaacag gttgtggtgc ctcctggagt gacaaactcc
tcttttcaag tgacatctca aaatgttgga caacttactg
tttatctaca tggaaatcac tccaatcaga ccgggtaggc
tggcctcagg gtgtgggggc ctcacgtgac aagaaggggc
ccgt

GenBank Accession No.: Y15928.1, human CTNS gene, exon 7 and flanking intronic regions, which provides the nucleic acid sequence (SEQ ID NO: 9):

ccttcataag cccagcctca gctcatcccg gtccccaaac
tcctttccag cccgaggata cgctttcttg tgatccgcag
cagcgccatt agcatcataa accaggtgat tggctggatc
tactttgtgg cctggtccat ctccttctac cctcaggtga
tcatgaattg gaggcggaaa aggtaacccc ctgggccgta
tgtgcaggct ctctcggggc ccctaggagc ag

GenBank Accession No.: Y15929.1, human CTNS gene, exon 8 and flanking intronic regions, which provides the nucleic acid sequence (SEQ ID NO: 10):

ccctgccagt cttcaccccc tgccctgtct tgtccctcca
ccccctgcag tgtcattggt ctgagcttcg acttcgtggc
tctgaacctg acaggcttcg tggcctacag tgtattcaac
atcggcctcc tctgggtgcc ctacatcaag gtacggcctt
gcctgcccta catctctgcc cacatggcgt ggtggcccgg

GenBank Accession No.: Y15930.1, human CTNS gene, exon 9 and flanking intronic regions, which provides the nucleic acid sequence (SEQ ID NO: 11):

tggtggcccg gctgcccctc accacccagc ttctcccacc
caccaaacag gagcagtttc tcctcaaata ccccaacgga
gtgaaccccg tgaacagcaa cgacgtcttc ttcagcctgc
acgcggttgt cctcacgctg atcatcatcg tgcagtgctg
cctgtatgag gtgagaccag ccctggcccc ccacaggcca
ccccagccaa cacccgccac

GenBank Accession No.: Y15931.1, human CTNS gene, exon 10 and flanking intronic regions, which provides the nucleic acid sequence (SEQ ID NO: 12):

cggcgtggcc tctgtgtggg tccacatctc tgccctcctc
tcgcccccag cgcggtggcc agcgcgtgtc ctggcctgcc
atcggcttcc tggtgctcgc gtggctcttc gcatttgtca
ccatgatcgt ggctgcagtg ggagtgatca cgtggctgca
gtttctcttc tgcttctcct acatcaagct cgcagtcacg
ctggtcaagt attttccaca ggtacctcca gggccctgtt
cacatggccg gtggcaggag aggtgagagc t

GenBank Accession No.: Y15933.1, human CTNS gene, exon 11 and flanking intronic regions, which provides the nucleic acid sequence (SEQ ID NO: 13):

ccgcccagcc ctcaccgccc tccgtctgta tgtccgtctg
tctggcccag gcctacatga acttttacta caaaagcact
gagggctgga gcattggcaa cgtgctcctg gacttcaccg
ggggcagctt cagcctcctg cagatgttcc tccagtccta
caacaacggt gagtcagcca gcgggctgct ggccaccctg
cggctggggc atcgggcg

In another aspect, the method of treating an inherited eye disease or disorder in a subject includes contacting cells expressing a protein associated with the particular disease or disorder (see Table 1) from the subject with a vector encoding a gene editing system that when transfected into the cells corrects a mutation (e.g., deletions, missense mutations, in-frame deletions and/or insertions) of the endogenous gene, and transplanting the transfected cells into the subject, thereby treating the inherited eye disease or disorder. In various embodiments, the gene editing system is selected from the group consisting of CRISPR/Cas, zinc finger nucleases, and transcription activator-life effector nucleases. The step of contacting may be performed ex vivo by first obtaining a sample of cells from the subject, transfecting the gene editing system into the sample of cells, and thereafter transplanting the transfected cells into the subject (e.g., into the eye of the subject), thereby treating the inherited eye disease or disorder. The sample of cells may be any cells expressing the protein associated with the inherited eye disease or disorder, such as, for example, blood cells or HSPCs of the subject.

In another aspect, the present invention provides a method of treating or ameliorating an inherited eye disease or disorder in a subject. The method includes transplanting a population of HSPCs into the subject (e.g., into the eye of the subject), wherein the HSPCs have been genetically modified by introduction of a transgene encoding a corresponding functional human protein, thereby treating the inherited eye disease or disorder. Thus, as described above, when the inherited eye disease or disorder is ocular cystinosis, the functional human lysosomal transmembrane gene is CTNS. In various embodiments, the HSPCs are isolated from the subject, such as from the blood or bone marrow of the subject.

While the present invention has been demonstrated with regard to cystinosis, it should be understood that the methods are applicable to any inherited eye diseases or disorders. Thus, this strategy turns HSPCs into intelligent and widespread delivery vehicles to obtain stable and sustained cross-correction after their differentiation into monocytes that enter the circulation and subsequently invade the peripheral tissues where they transform into tissue resident macrophages. These macrophages, through a variety of mechanisms including, but not limited to, the formation of tunneling nanotubes, vesicular release, and direct cell-cell adhesion, transfer their lysosomes, which carry the respective protein to diseased peripheral cells. As such, this work demonstrates the development of a HSPC gene therapy strategy for treating inherited eye diseases or disorders.

The following examples are intended to illustrate but not limit the invention.

### EXAMPLE 1

### Preclinical Model of Cystinosis for Testing Therapeutic Approaches

Stem cell therapeutic approaches have been tested on the mouse model of cystinosis, the Ctns^{-/-} mice. This murine model was engineered to produce defective cystinosin, and is thus unable to properly transport cystine out of the lysosomes. The defect results in accumulation of cystine and formation of cystine crystals, pathognomonic of cystinosis. Cystine accumulation is present from birth and increases with age. Usefully, the Ctns^{-/-} mice developed ocular defects with corneal cystine crystal depositions and thyroid dysfunction similar to those observed in affected patients.

### EXAMPLE 2

### Long-term Effect of HSC Transplantation in Ctns^{-/-} mice

Eye analysis: GFP⁺WT HSC transplantation led to the long-term preservation of the eyes in Ctns^{-/-} mice. Abundant GFP+ bone marrow-derived cells were detected within the cornea but also in the sclera, ciliary body, retina, choroid, and lens in the treated mice. To quantify cystine crystals within the cornea, in vivo confocal microscopy (IVCM) in live mice was performed. While Ctns^{-/-} mice with low level of engraftment (<50%; LOW; n=5) presented a partial reduction of crystal counts, the mice with high engraftment levels (>50%; HIGH; n=5) exhibited almost a complete resolution of crystals from the epithelial layer to the middle stroma (100% to 72% clearance, respectively). One-year post-transplantation, HSC-treated Ctns^{-/-} mice exhibited normal corneal thickness and structure and normal intraocular pressure. This work was the first demonstration that transplanted HSCs could rescue corneal defects and brings new perspectives for ocular regenerative medicine.

Although the invention has been described with reference to the above examples, it will be understood that modifications and variations are encompassed within the spirit and scope of the invention. Accordingly, the invention is limited only by the following claims.

1. A method of treating an inherited eye disease or disorder in a subject comprising:
introducing a corresponding functional human protein associated with the inherited eye disease or disorder into hematopoietic stem and progenitor cells (HSPCs) of the subject; and
transplanting the HSPCs into an eye of the subject, thereby treating the inherited eye disease or disorder.

2. The method of claim 1, wherein the inherited eye disease or disorder is ocular cystinosis and the corresponding functional human protein is cystinosin (CTNS).

3. The method of claim 1, wherein the step of introducing comprises contacting a vector comprising a polynucleotide encoding the functional human protein associated with the inherited eye disease or disorder and a functional promoter with the HSPCs and allowing expression of the functional human protein associated with the inherited eye disease or disorder.

4-5. (canceled)

6. The method of claim 1, wherein the vector is a viral vector selected from the group of a lentiviral, adenoviral, or an AAV vector.

7. The method of claim 6, wherein the vector is a self-inactivating (SIN)-lentivirus vector or pCCL-CTNS.

8. (canceled)

9. The method of claim 1, wherein the step of introducing is performed ex vivo.

10. (canceled)

11. The method of claim 1, wherein the step of transplanting comprises intracameral injection or intravitreal injection.

12. A method of treating or ameliorating an inherited eye disease or disorder in a subject comprising:
isolating hematopoietic stem and progenitor cells (HSPCs) from blood or bone marrow of the subject;
introducing a functional human gene into the HSPCs, wherein the gene encodes a protein corresponding to the inherited eye disease or disorder; and
transplanting the HSPCs back into an eye of the subject, thereby treating or ameliorating the lysosomal protein disease or disorder.

13. (canceled)

14. The method of claim 12, wherein the HSPCs are CD34+ cells.

15-18. (canceled)

19. The method of claim 12, wherein the level of cystine in the eye of the subject is reduced following treatment.

20. The method of claim 12, wherein the subject was on cysteamine therapy prior to treatment.

21. (canceled)

22. The method of claim 12, wherein cystine or cystine crystals are measured in the eye prior to and/or following treatment.

23. The method of claim 22, wherein cystine levels are measured prior to, during and/or following treatment.

24. The method of claim 22, wherein cystine levels are measured in biological samples obtained from the subject.

25. (canceled)

26. The method of claim 22, wherein cystine crystals are measured using in vivo confocal microscopy.

27. A method of treating or ameliorating an inherited eye disease or disorder in a subject comprising:
producing a functional human gene associated with the inherited eye disease or disorder in the subject using a gene editing system.

28-33. (canceled)

34. A method of treating or ameliorating an inherited eye disease or disorder in a subject comprising contacting cells expressing a defective protein associated with the inherited eye disease or disorder from the subject with a vector encoding a gene editing system that, when transfected into the cells, corrects a mutation of an endogenous gene encoding the defective protein, thereby treating the inherited eye disease or disorder.

35. (canceled)

36. The method of claim 34, wherein the gene editing system is selected from the group consisting of CRISPR/Cas, zinc finger nucleases, and transcription activator-life effector nucleases.

37. (canceled)

38. The method of claim 34, wherein the step of contacting comprises obtaining a sample of cells from the subject, transfecting the gene editing system into the sample of cells, and thereafter, transplanting the transfected cells into the subject.

39-40. (canceled)
